(19) United States
(12) Patent Application Publication
(10) Pub. No.: US 2026/0306554 A1
(43) Pub. Date: Oct. 8, 2026

(54) RECOMBINANT EXPRESSION VECTOR FOR INTERFERON-BETA SECRETION AND ATTENUATED SALMONELLA STRAIN TRANSFORMED BY MEANS OF SAME

(71) Applicants: THE INDUSTRY & ACADEMIC COOPERATION IN CHUNGNAM NATIONAL UNIVERSITY (IAC), Daejeon (KR); KOREA INSTITUTE OF SCIENCE AND TECHNOLOGY, Seoul (KR)

(72) Inventors: Hyo Jin LEE, Daejeon (KR); Heung Jin JEON, Daejeon (KR)

(73) Assignee: THE INDUSTRY & ACADEMIC COOPERATION IN CHUNGNAM NATIONAL UNIVERSITY (IAC), Daejeon (KR)

(21) Appl. No.: 18/998,406

(22) PCT Filed: Jul. 25, 2023
(86) PCT No.: PCT/KR2023/010767

§ 371 Date: Jan. 24, 2025

(30) Foreign Application Priority Data
Jul. 25, 2022 (KR) 10-2022-0092017

(51) Int. Cl. A61K 35/74; A61K 38/00; A61P 35/00; C07K 14/195; C07K 14/57; C12N 1/20; C12N 15/74; C12R 1/42

(52) U.S. Cl. CPC A61K 35/74; A61P 35/00; C07K 14/195; C07K 14/57; C12N 1/20; C12N 15/74; A61K 38/00; C12R 2001/42

(57) ABSTRACT

The present disclosure relates to a recombinant expression vector for secretion of an IFN-β protein and an attenuated Salmonella strain transformed therewith, and provides: a recombinant expression vector comprising an flgM gene, an IFN-β gene, and an flhDC gene; an attenuated Salmonella strain transformed therewith; and a pharmaceutical composition for cancer treatment, comprising the attenuated Salmonella strain as an active ingredient.

### TECHNICAL FIELD

The present disclosure relates to a recombinant expression vector for secretion of interleukin-beta and an attenuated Salmonella strain transformed therewith. The present application claims priority to Korean Patent Application No. 10-2022-0092017, filed on Jul. 25, 2022, in the Korean Intellectual Property Office, the disclosure of which is incorporated herein by reference.

### BACKGROUND ART

Cancer is a life-threatening disease caused by cells that do not stop proliferating, but invade surrounding tissues and eventually destroy normal cells. As the regulatory ability of the human body declines with age, the human body becomes vulnerable to cancer, which has been the leading cause of death in aging societies for 10 years since 2007, more than doubling the second leading cause of death, heart disease.

The most effective response system for treating disease is to strengthen immunity, but cancer cells are not foreign invaders, and therefore they evade the body's immune response without triggering it. Certain viruses or bacteria are more likely to infect cancer cells than normal cells, and such infected cancer cells may be targeted for the attack of immune cells. Accordingly, anticancer treatment methods have been proposed that intentionally use specific viral or bacterial infections to stimulate immune responses in the human body to fight against cancer cells. Infectious bacteria such as Shigella, Vibrio cholerae, and pathogenic E. coli invade intestinal cells, but do not reach the liver and spleen, which are important organs for triggering an immune response. In contrast, Salmonella may invade the spleen and liver through lymph nodes and stimulate a systemic immune response. Specifically, according to Leschner et al. (J. Mol. Med. 2010, 88, 763-773), CT26 tumor-bearing mice were intravenously injected with fluorescent Salmonella, and infection pathways were tracked over time. As a result, it was observed that, immediately after infection, the whole body of the mice was infected through the blood, Salmonella accumulated in the spleen and liver 20 minutes after infection, and Salmonella accumulated intensively only in tumor tissue 24 hours after infection.

However, Salmonella as a representative bacterium that causes food poisoning can cause life-threatening sepsis through infection, and thus it is too pathogenic to be used directly for cancer treatment. A research team at Yale University in the United States announced that, through genetic modification of Salmonella, Salmonella could be attenuated by removing its toxicity while maintaining its tumor-attacking properties, and that injection of the attenuated Salmonella may suppress tumors by inducing immune stimulation. However, an attenuated Salmonella is not only less viable and less able to cause immunity, but there is also a risk that mutations can cause such an attenuated strain to convert to wild-type Salmonella and cause sepsis. In addition, due to a lack of research and development funding, most anticancer treatments using attenuated Salmonella have been suspended or put on hold in Phase I in clinical trials.

The potential for causing sepsis associated with cancer treatment using bacteria such as Salmonella is a critical issue that must be overcome, and in addition, the stimulation of the immune response must also be actively achieved. Accordingly, research is mainly focused on developing strains that can additionally express or suppress substances beneficial to anticancer treatment, along with attenuation and immune activation, and using these strains in anticancer treatment. KR Patent Registration No: 10-0852687 discloses a Salmonella strain expressing tumor necrosis factor alpha and an anticancer therapeutic composition comprising the same, wherein a tumor necrosis factor alpha protein vector is transfected into an attenuated Salmonella strain, and KR Patent Registration No: 10-1750007 discloses a solid cancer therapeutic agent using an attenuated Salmonella strain in which L-asparaginase can be selectively expressed at a tumor site.

Interferon (IFN) is one of the major cytokines playing an important role in immunity, and is known to have strong anticancer effects. IFN-beta (IFN-β) is a type 1 interferon that is known to directly inhibit tumor growth by interacting with p53 in the tumor environment. In addition, interferon-treated neutrophils are shown to upregulate PD-L1 and inhibit T-cell proliferation. IFN-3 binds to IFNAR1 and IFNAR2, which are subunits of the type I INF receptor to transmit signals through TYK2 and JAK2, which in turn phosphorylates the STAT family members (e.g., STAT1, STAT2, STAT3, etc.) and activates downstream functions to stimulate anti-tumor activity. For example, a vesicular stomatitis virus expressing IFN-β has been shown to enhance the antitumor immune response in a mouse model of non-small cell lung cancer (NSCLC) (Kim Y, Lee D, Lee J, Lee S, Lawler S (2019) Role of tumor-associated neutrophils in regulation of tumor growth in lung cancer development: A mathematical model. PLoS ONE 14 (1): e0211041).

In this regard, the inventors of the present disclosure prepared a recombinant expression vector and an attenuated Salmonella strain transformed with the recombinant expression vector, the recombinant expression vector including: an flgM gene; an IFN-3 gene; and an flhDC gene, and confirmed excellent cancer therapeutic effects of the recombinant expression vector, thereby completing the present disclosure.

The information set forth in this Background Art section is intended solely to enhance understanding of the background of the present disclosure, and may not include information that constitutes prior art already known to a person skilled in the art to which the present disclosure pertains.

### DISCLOSURE OF INVENTION

### Technical Problem

An aspect is to provide a recombinant expression vector including: an flgM gene; an IFN-β gene; and an flhDC gene, wherein the flgM gene, the IFN-β gene, and the flhDC gene are operably linked to an inducible promoter.

Another aspect is to provide an attenuated Salmonella strain transformed with the recombinant expression vector.

Another aspect is to provide a pharmaceutical composition for cancer treatment, including the attenuated Salmonella strain as an active ingredient.

### Solution to Problem

An aspect is to provide a recombinant expression vector including: an flgM gene; an IFN-β gene; and an flhDC gene, wherein the flgM gene, the IFN-β gene, and the flhDC gene are operably linked to an inducible promoter.

The present disclosure relates to a recombinant expression vector in which the flgM gene and the IFN-β gene are directly linked or are linked to each other via a linker, such that the recombinant expression vector includes a gene encoding a fusion protein of flgM and IFN-β. Hereinafter, in the present specification, a fusion protein of the flgM and the IFN-3 is named “flgM-IFN-β”.

In the present specification, the term “flgM” refers to a protein (or a gene encoding such a protein) that inhibits the a factor, which is responsible for recruiting a RNA polymerase required for late flagellar transcription in a type III secretion system.

The “type III secretion system” refers to a pathogenic substance delivery system developed in Gram-negative bacteria. It is a multi-protein complex in the form of a syringe-shaped device that injects pathogenic active proteins directly into the cytoplasm through the membrane of a host cell (Mota L J et al, Ann Med. (2005); 37(4):234-249). In the type III secretion system, the flgM protein remains in the cytoplasm during the formation of the hook-basal body which is a basic structure capable of forming flagella on the cell membrane, and acts as an anti-σ²⁸ factor that inhibits the transcription of class III genes, such as flagellin subunit FliC or stator protein MotAB. Subsequently, after completion of the hook-basal body structure, a change in substrate specificity occurs simultaneously within an flagellar secretion system, causing flgM to be secreted out of the cell through the central pathway of the hook. Then, inside the cell, σ²⁸-dependent transcription begins. A secretion mechanism of flgM through the flagella formation process in the type III secretion system is shown in FIG. 3.

According to an embodiment, the present disclosure is to manufacture the recombinant expression vector including the flgM gene and the IFN-β gene, which is a protein to be secreted, so that the strain may secrete IFN-β outside the strain together with flgM through the type III secretion system. The genetic information for IFN-β can be obtained from known databases such as GenBank of the National Center for Biotechnology Information (NCBI).

In the present specification, the term “flhDC” refers to a major regulator of an flagellin operon gene, and flhDC can regulate the expression of the flgM gene. In an embodiment, by including the flhDC gene in addition to the flgM gene and the IFN-β gene, the recombinant expression vector may contribute to increasing the expression level or secretion level of the flgM-IFN-β.

In the present specification, the term “gene” should be considered in its broadest sense, and a gene may be able to encode a structural protein or a regulatory protein. Here, the regulatory protein may include a transcription factor, a heat shock protein, or a protein involved in DNA/RNA replication, transcription, and/or translation.

In the present specification, the term “genetic structure” refers to an aggregate, functional unit, or a construct including the genetic information of a protein of interest, and may be considered in its broadest sense. The genetic structure may be, for the purpose of the present disclosure, intended to enhance the secretion of the flgM-IFN-β from the attenuated Salmonella strain, and may refer to, for example, a DNA insert to which the flgM gene, a linker gene, and the IFN-β gene are operably linked.

In the present specification, the term “vector” refers to a DNA product containing a nucleotide sequence encoding a target polynucleotide operably linked to suitable regulatory sequences such that the target polynucleotide can be expressed in a suitable host. The regulatory sequences may include a promoter capable of initiating transcription, an optional operator sequence for regulating such transcription, a sequence encoding a suitable mRNA ribosome-binding site, and a sequence regulating termination of transcription and translation. Once transformed into a suitable host, the vector may be replicated or function regardless of the host genome, or may be integrated into the genome itself. The vector may be any one capable of replication in a host cell. For example, the vector may be a plasmid, cosmid, virus, or bacteriophage that is either native or recombinant. The vector may include a selection marker for selecting a host cell containing the vector. The selection marker is for selecting a cell transformed with the vector, and markers that confer a selectable phenotype, such as drug resistance, nutrient requirement, resistance to a cytotoxic agents, or expression of surface proteins, may be used. For example, the marker may be one or more selection markers selected from the group consisting of ampicillin, neomycin, puromycin, hygromycin, and zeocin. The vector may include genes involved in replication and/or copy number control, such as a replication origin, a promoter, or the like, and may include, although not limited thereto, a restriction enzyme site or the like. In addition, appropriate restriction enzymes may be placed at the front and back ends of the genetic structure of the present disclosure to enable introduction of the genetic structure of the present disclosure into the vector.

In the present specification, the term “polynucleotide” may be in the form of RNA or DNA, and DNA may include cDNA and synthetic DNA. DNA may be single-stranded or double-stranded. In the case of a single strand, it may be a coding strand or a non-coding (antisense) strand, and a coding sequence may, as a result of degeneracy or redundancy of the genetic code, may encode the same polypeptide.

The polynucleotide may also include a variant of the polynucleotide described herein, wherein the variant of the polynucleotide may be a naturally occurring allelic variant of the polynucleotide or a non-naturally occurring variant of the polynucleotide. The allelic variant may be an alternate form of a polynucleotide sequence that may have substitution, deletion, or addition of one or more nucleotides, without substantially changing the function of a polynucleotide to be encoded. It is well known in the art that a single amino acid may be encoded by one or more nucleotide codons and that the polynucleotide may be easily modified to prepare an alternate polynucleotide encoding the same peptide.

In an embodiment, by including all the flgM gene, the IFN-β gene, and the flhDC gene, the recombinant expression vector may contribute to increasing the expression level or secretion level of the flgM-IFN-β. For example, when the recombinant expression vector further includes the flhDC gene in addition to the flgM gene and the IFN-β gene in gene, the expression level or secretion level of the flgM-IFN-β may be increased.

In the present specification, the term “inducible promoter” refers to a promoter capable of switching the operation of a gene linked by an inducer, and examples thereof may include an ara promoter, a tac promoter, a lac promoter, a lacUV5 promoter, a Ipp promoter, a pLλ promoter, a pRλ promoter, a rac5 promoter, an amp promoter, a recA promoter, a SP6 promoter, a trp promoter, a T7 promoter, a pBAD promoter, a Tet promoter, a trc promoter, a pepT promoter, a sulA promoter, a poll 1(dinA) promoter, a ruv promoter, a uvrA promoter, a uvrB promoter, a uvrD promoter, a umuDC promoter, a lexA promoter, a cea promoter, a caa promoter, a recN promoter, a pagC promoter, a hip promoter, an ansB promoter, or a pflE promoter. In an embodiment, the promoter may be an ara promoter.

In the present specification, the expression “operably linked” may refer that nucleotide sequences are linked on a single nucleic acid fragment such that one function is affected by the other.

In an embodiment, expression of the flgM gene, the IFN-β gene, and the flhDC gene may be regulated by the same inducible promoter. In an embodiment, the promoter may be an ara promoter.

In an embodiment, due to the recombinant vector in which the expression of the flgM gene, the IFN-β gene, and the flhDC gene is regulated by the same inducible promoter, the expression ability or secretion ability of the flgM-IFN-β may be enhanced.

In an embodiment, the recombinant expression vector may include the flgM gene consisting of a polynucleotide sequence of SEQ ID NO: 1, the IFN-β gene consisting of a polynucleotide sequence of SEQ ID NO: 3, and the flhDC gene consisting of a polynucleotide sequence of SEQ ID NO: 7. In an embodiment, the recombinant expression vector may include a polynucleotide sequence of SEQ ID NO: 6. In an embodiment, the recombinant expression vector may have an enhanced ability to express or secrete the flgM-IFN-β by including the polynucleotide sequence of SEQ ID NO: 3.

In an embodiment, the flgM gene and the IFN-β gene may be linked to each other via a linker.

In the present specification, the term “linker” refers to a polypeptide chain used to link a heterologous domain to a protein of interest, or a gene encoding the polypeptide chain. A linker peptide may have 2 to 50 amino acids in length. For example, although not limited thereto, the linker peptide may have, in length, 2 to 40 amino acids, 2 to 30 amino acids, 2 to 20 amino acids, 2 to 15 amino acids, 2 to 10 amino acids, 2 to 5 amino acids, 5 to 50 amino acids, 5 to 40 amino acids, 5 to 30 amino acids, 5 to 20 amino acids, 5 to 15 amino acids, or 5 to 10 amino acids. For example, the linker peptide may be, although not limited thereto, at least one selected from the group consisting of (GₗSₘ)ₙ (where l, m, and n are each 2), (GₗSₘ)ₙ-Hₚ-(GₗSₘ)ₙ (where l, mm and n are each ≥1, H≥6), (G₄S)ₐ(EAAAK)_{b}(G₄S)ₐ (where a and b are each an integer from 1 to 4), (G₄S)ₚ(EAAAK)_{q} (where p and q are each an integer from 1 to 4), (EAAAK)ₓ(G₄S)_{y} (where x and y are each an integer from 1 to 4), A(EAAAK)₄ALEA(EAAAK)₄A, (GSSGGS)ᵢ (where i is an integer from 1 to 4), KESGSVSSEQLAQFRSLD, EGKSSGSGSESKST, GSAGSAAGSGEF, (EAAAK)ₖ (where k is an integer from 1 to 5), CRRRRRREAEAC, GGGGGGGG, GGGGGG, AEAAAKEAAAAKA, PAPAP, VSQTSKLTRAETVFPDV, PLGLWA, TRHRQPRGWE, AGNRVRRSVG, RRRRRRRR, GGSSHHHHHHSSGG, GGSSHHHHHHSSGGLVPRGSH, and GSSGGSGSSGGSGGGDEADGSRGSQKAGVDE. In an embodiment, the linker peptide may be GGSSHHHHHHSSGG or GGSSHHHHHHSSGGLVPRGSH.

In an embodiment, the flgM gene and the IFN-β gene may be linked via a linker including a polynucleotide sequence of SEQ ID NO: 4 or 5.

The recombinant expression vector according to an embodiment may have an enhanced ability to express or secrete the flgM-IFN-β by linking the flgM gene and the IFN-β gene through a gene (SEQ ID NO: 5) encoding the GGSSHHHHHHSSGG linker peptide.

Another aspect is to provide an attenuated Salmonella strain transformed with the recombinant expression vector. Regarding the attenuated Salmonella strain, the same terms or elements as those already mentioned are as described above.

In the present specification, the term, “transformation” refers to introducing a vector including a polynucleotide encoding a target protein into a host cell to enable the expression of the protein encoded by the polynucleotide within the host cell. For example, the polynucleotide may be introduced in the form of an expression cassette into a host cell, or may be introduced in its own form into a host cell, so as to be operably linked to a sequence necessary for the expression in the host cell. However, the polynucleotide is not limited thereto.

In the present specification, the term “attenuated” refers to artificially weakening the virulence of a living pathogen. In detail, it means that, by mutating genes involved in the essential metabolism of the pathogen, only the immune system is stimulated without causing a disease in the body, thereby inducing immunity. Genes causing attenuation of Salmonella are well known in the art, and the attenuated Salmonella strain may be, for example, one in which the function of one or more genes selected from the group consisting of aroA, aroC, aroD, aroE, asd, Rpur, rcsB, htrA, ompR, ompF, ompC, galE, cya, crp, cyp, phoP, phoQ, rfaY, dksA, hupA, sipC, clpB, clpP, clpX, pab, nadA, pncB, pmi, rpsL, hemA, rfc, poxA, galU, cdt, pur, ssa, guaA, guaB, fliD, flgK, flgL, relA, and spoT has been lost. To prevent reversion to the wild type upon in vivo application and to further attenuate virulence, the function of two or more of the genes above may be lost.

The attenuated Salmonella strain may have anticancer activity by targeting a tumor site and inducing an immune response. The attenuated Salmonella strain according to an embodiment may be transformed with the recombinant expression vector, and it was confirmed that the expression level and secretion level of the flgM-IFN-β were significantly enhanced, thereby confirming that an agent including the Salmonella strain exhibits an excellent anticancer effect when administered to an individual.

The attenuated Salmonella strain according to an embodiment may be one in which the functions of aroA, aroD, asd, and galE genes have been lost, or one in which the functions of aroA, aroD, asd, rcsB, and galE genes have been lost. In an embodiment, when the attenuated Salmonella strain in which the functions of aroA, aroD, asd, rcsB, and galE genes have been lost is transformed with the recombinant expression vector according to an embodiment, it was confirmed that the ability to express and secrete the flgM-IFN-β were significantly increased.

The attenuated Salmonella strain according to an embodiment may be one in which the expression of an attenuated gene is regulated by a second inducible promoter. That is, the loss of function of a specific gene may be regulated by a second inducible promoter. In detail, the attenuated Salmonella strain may further include a second inducible promoter upstream of the attenuated gene to enable switching the attenuation of the strain. For example, the attenuated Salmonella strain may be injected in an attenuated state into a subject to target a tumor site, and then, the expression of the attenuated gene may be induced by operation of the second inducible promoter, thereby inducing high immune activity and exhibiting cancer treatment more effectively.

In an embodiment, the attenuated Salmonella strain may have lost a galE gene, and loss of function of the galE gene may be covered by switch-of-function of a tetRA promoter. Therefore, after the attenuated Salmonella strain is injected in an attenuated into a subject and targeted a tumor site, the expression of the galE gene is induced by doxycycline, leading to induction of high immune activity of the Salmonella strain. In the present specification, such expression regulation of the attenuated Salmonella strain is expressed as “galE:tetRA”.

The attenuated Salmonella strain according to an embodiment may be a BRD509 asd rcsB galE:tetRA-strain, and it is accordingly confirmed that the attenuated Salmonella strain has excellent ability to express or secrete the flgM-IFN-β.

In an embodiment, the attenuated Salmonella strain may be one deposited with Accession No. KCTC15509BP.

Another aspect provides a pharmaceutical composition for preventing or treating cancer, including the attenuated Salmonella strain as an active ingredient. Regarding the pharmaceutical composition, the same terms or elements as those already mentioned are as described above.

“Cancer” which is a target disease to be prevented or treated by the composition of the present disclosure is meant to be a collective term for diseases caused by cells that have aggressive characteristics of dividing and growing beyond normal growth limits, invasive characteristics of infiltrating surrounding tissues, and metastatic characteristics of spreading to other parts of the body. The cancer may be, for example, a solid cancer. Preferably, the cancer may be expanded without limitation as long as it is a tumor including cancer tissues or cancer cells in which the cancer-targeting ability of the Salmonella strain can be exerted. For example, the type of cancer may be, although not limited thereto, any one selected from the group consisting of head and neck cancer, skin cancer, pancreatic cancer, colorectal cancer, colon cancer, stomach cancer, liver cancer, brain cancer, breast cancer, thyroid cancer, bladder cancer, esophageal cancer, uterine cancer, and lung cancer.

The composition of the present disclosure may be prepared by a method known in the pharmaceutical field for use as a drug fur preventing or treating cancer, and may be used on its own or in a mixture with a pharmaceutically acceptable carrier, an excipient (e.g., a forming agent), a diluent, and the like.

In the present specification, a “pharmaceutically acceptable carrier” may be determined in part by a particular composition being administered, as well as in part by a particular method used to administer the composition. Therefore, suitable formulations of the composition may be very diverse, and the composition may be, for example, formulated for parenteral (i.e., intramuscular, intradermal, or subcutaneous) administration or nasopharyngeal (i.e., intranasal) administration. In general, formulations for parenteral administration may include sterilized aqueous solution or non-aqueous solution, suspensions, and emulsions. Examples of the non-aqueous solvent may include propylene glycol, polyethylene glycol, plant oil such as olive oil, and injectable organic esters such as ethyl oleate. An aqueous carrier may include water, alcohol/aqueous solutions, emulsions, or suspensions, in addition to saline and buffered media. Parenteral vehicles may include sodium chloride solution, Ringer's dextrose, dextrose and sodium chloride, lactated Ringer's solution, fixed oils. Preservatives and other additives may be also present in the form of, for example, antibacterial agents, antioxidants, chelating agents, inert gases, and the like.

A dosage of the active ingredient according to the present disclosure may be appropriately selected depending on the degree of absorption of the active ingredient in the body, the type of formulations, the age, gender, and condition of a patient, the degree of symptoms, and the like, and the active ingredient, and the active ingredient may be administered once a day or divided into several times. A typical dosage is 0.001 mg/kg·day to 10 g/kg·day.

Another aspect provides a method of preventing or treating cancer, the method including administering to a subject an anticancer pharmaceutical composition including the attenuated Salmonella strain as an active ingredient in an amount effective for preventing or treating cancer. Regarding the method of preventing or treating cancer, the same terms or elements as those already mentioned are as described above.

The subject may be a mammal. The mammal may be a human, a dog, a cat, a cow, a goat, or a pig.

The administration may be done by any common route as long as the composition can reach a target tissue. For example, the administration may be done by routes such as eye drop administration, intraperitoneal administration, intravenous administration, intramuscular administration, subcutaneous administration, intradermal administration, transdermal patch administration, oral administration, intranasal administration, intrapulmonary administration, and rectal administration, and specifically, the administration may be done as intended by a route of eye drop administration.

In the present specification, the term “treatment” or “treating” or “alleviating” or “ameliorating” is used interchangeably. These terms refer to methods of obtaining beneficial or desired results including, but not limited to, therapeutic benefits and/or prophylactic benefits. Therapeutic benefit means any therapeutically relevant improvement in or effect on one or more diseases, illnesses, or symptoms under treatment. For prophylactic benefits, the composition may be administered to a subject at risk of developing a particular disease, illness, or symptom, or to a subject reporting one or more physiological symptoms of a disease, even though a disease, illness or symptom has not yet manifested.

As used in the present specification, the term “effective amount” or “therapeutically effective amount” refers to an amount of an agent that is sufficient to cause a beneficial or desired result. The therapeutically effective amount may vary depending on one or more of a subject and pathological conditions being treated, the weight and age of a subject, severity of a pathological condition, administration methods, etc., which may be readily determined by a person skilled in the art. In addition, the term is applied to a dose that will provide an image for detection by any one of imaging methods described herein. A specific dose may vary depending on one or more of a particular agent chosen, a dosing regimen to be followed, whether it is administered in combination with other compounds, timing of administration, a tissue to be imaged, and a physical delivery system carrying the tissue.

### Advantageous Effects of Invention

According to the recombinant expression vector of an aspect, it was confirmed that the expression and secretion of the flgM-IFN-β were significantly enhanced in the attenuated Salmonella strain transformed with the recombinant expression vector.

Also, according to the recombinant expression vector of an aspect, it was confirmed that excellent anticancer effects and excellent stability were exhibited upon administration of a formulation including the attenuated Salmonella strain transformed with the recombinant expression vector.

### BRIEF DESCRIPTION OF DRAWINGS

FIG. 1 is a schematic diagram showing a process of tumor suppression of IFN-β.

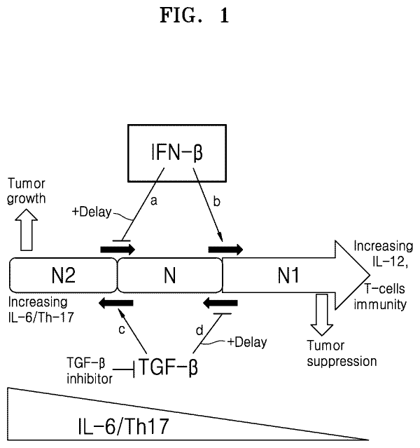

FIG. 2 is a schematic diagram showing a process of extracellular secretion of flgM-IFN-β, using a type III secretion system.

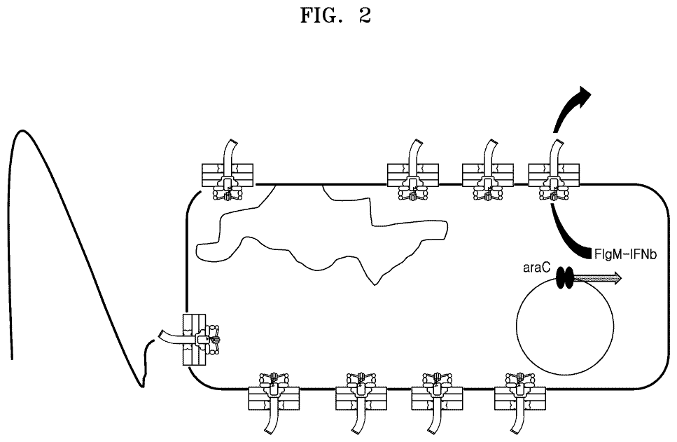

FIG. 3 is a schematic diagram showing an flgM secretion mechanism using a flagella formation process in a type III secretion system.

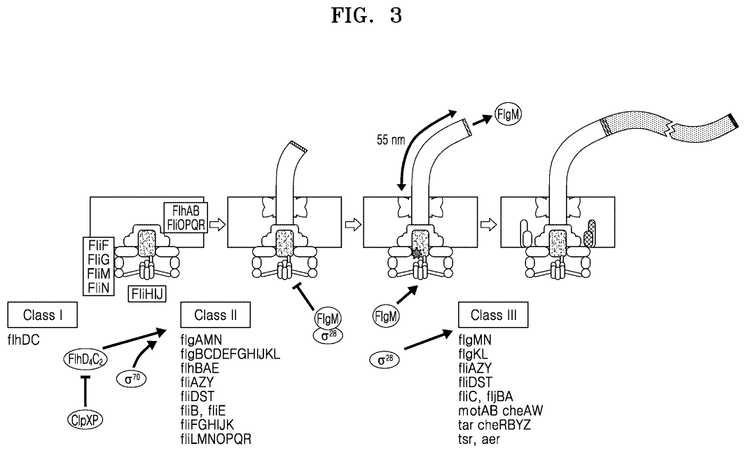

FIG. 4 is a diagram showing a cleavage map of an flgM-IFN-β-flhDC-pBAD18-asd+plasmid.

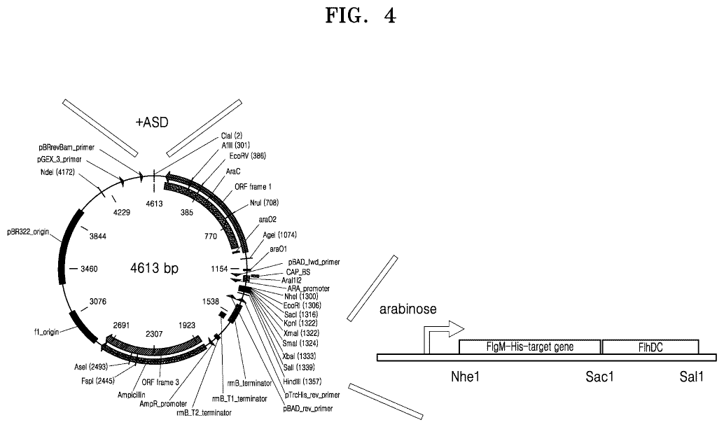

FIG. 5 shows results of western blotting, confirming a secretion level of an flgM-IFN-β protein of a strain according to an embodiment, wherein, in FIG. 5, (a) shows results of Comparative Example 1, and (b) shows results of Comparative Example 2.

FIG. 6 shows results of western blotting, confirming a secretion level of an flgM-IFN-β protein of a strain according to an embodiment, wherein, in FIG. 6, (a) shows results of Comparative Example 3, and (b) shows results of Example 1.

FIG. 7 is a schematic diagram illustrating a process of treating a tumor mouse model with a strain according to an embodiment.

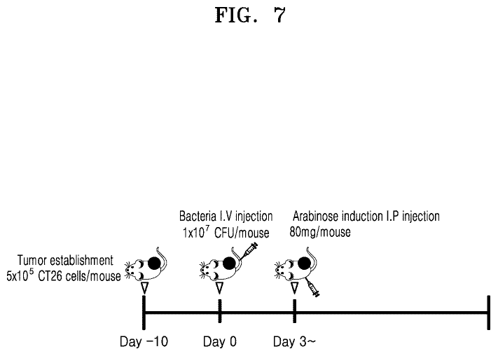

FIG. 8 is a graph showing changes in tumor volume in a mouse when treated with a strain according to an embodiment (CTRL: control group, Bacteria: arabinose-untreated group, Induction: arabinose treated group).

FIG. 9 is a graph showing changes in body weight of a mouse when treated with a strain according to an embodiment (CTRL: control group, Bacteria: arabinose-untreated group, Induction: arabinose treated group).

### BEST MODE FOR CARRYING OUT THE INVENTION

### Mode for the Invention

Hereinafter, preferable examples are presented to help understanding of the present disclosure. However, examples below are only presented only for easier understanding of the present disclosure, and the contents of the present disclosure are not limited by these examples.

### Example 1. Preparation of Plasmid for IFN-β Secretion and Attenuated Salmonella Strain Transformed Therewith

### 1-1. Preparation of Insert DNA

To prepare a plasmid for secretion of IFN-β, a genetic structure in which an flgM gene and an IFN-β gene are linked was prepared.

Specifically, the flgM DNA sequence of wild-type Salmonella (flgM anti-sigma-28 factor FlgM [Salmonella enterica subsp. enterica serovar Typhimurium str. LT2] Gene ID: 1252690, Locus tagSTM1172 NC_003197.2 (1257036 . . . 1257341, complement)) and a DNA sequence obtained through codon optimization for IFN-β were linked via a linker to prepare a genetic structure. To increase the expression of the flgM gene, a shine dalgarno sequence was inserted upstream of the flgM gene. Also, considering cloning, an Nhe I site (GCTAGC) was inserted at the front and a Sac I site (GAGCTC) was inserted following the stop codon of the IFN-β. Through the gene synthesis service of Bionics Co. Ltd., the genetic structure including the flgM, the linker, and the IFN-β was prepared, which was then treated with 10 U of each of the Nhe I enzyme and the Sac I enzyme at 37° C. for 2 hours. Afterwards, electrophoresis was performed thereon, and DNA of about 900 bp was identified by using a gel extraction kit (by Qiagen Co., Ltd.) to obtain flgM-linker-IFN-β insert DNA.

Afterwards, to obtain flhDC insert DNA, PRC was performed by using the genomic DNA of wild-type Salmonella LT2 as a template and flhD-Sac1-F and flhC-Sal1-r shown in Table 1 as primers, and a PCR product including the flhDC gene was obtained. The specific PCR conditions are as follows: 5 μl of 10× buffer, 10 μl of 5×Q solution, 7.5 μl of 2 mM dNTPs, 2.5 μl of 20 μm flhD-sac1-f primer, 2.5 μl of 20 μm flhC-Sal1-r primer, 1 μl of Taq polymerase, 2 μl of 10 ng/μl genomic DNA, and 19.5 μl of water were mixed; and PCR was performed thereon by using Qiagen system at 95° C. for 5 minutes, followed by 30 cycles of PCR at 94° C. for 30 seconds, at 49° C. for 30 seconds, and at 72° C. for 1 minute, and then at 72° C. for 10 minutes. 1 μg of the PCR fragment purified by a PCR purification kit was reacted with 10 U of each of sac and Sal1 at 37° C. for 2 hours, and the reaction product was then purified by a PCR purification kit to prepare flhDC insert DNA (SEQ ID NO: 7).

TABLE 1

| | | SEQ |
|---|---|---|
| Name | Sequence information | ID NO: |
| flhD- | gatcgatcgagctcaggaggtttgatcctatgg | 8 |
| Sac1-f | gaacaatgcatacatccgagttgct | |
| flhC- | gatcgatcgtcgacttaaacagcctgttcgatc | 9 |
| Sal1-r | tgttcatccagcagtt | |

### 1-2. Preparation of Plasmid

Next, a pBAD18 asd+plasmid was used as a vector and reacted with 10 U of each enzyme, Nhe I and Sac I, at 37° C. for 2 hours. Then, the reaction product was purified by a PCR purification kit to obtain each vector DNA. The asd gene inserted into the plasmid may consist of the nucleotide sequence of SEQ ID NO: 10. Then, each vector DNA was ligated with the flgM-linker-IFN-β insert DNA at 25° C. for 30 minutes, and transformed into DH5a competent cells. Next, the transformed cells were spread onto a LB ampicillin (amp) solid medium and cultured at 37° C., and colonies having antibiotic resistance were selected. Six candidate groups were selected from the selected colonies and reacted with 10 U of each of Nhe I and Sac I at 37° C. for 1 hour. Then, generation of bands of about 900 bp was confirmed through electrophoresis. In addition, the nucleotide sequence analysis was performed on the candidate groups for the plasmid to confirm the insertion of the flgM, the linker, and the IFN-β gene.

1 μg of each plasmid was reacted with 10 U of Sac I and Sal I at 37° C. for 2 hours, and the reaction product was purified by a PCR purification kit, thereby completing the preparation of vector DNA. Then, each vector DNA was ligated with the flhDC insert DNA at 25° C. for 30 minutes and transformed into DH5a competent cells. The transformed cells were spread onto a LB amp solid medium and cultured at 37° C., and colonies having antibiotic resistance were selected. Six candidate groups were selected from the selected colonies and reacted with 10 U of each of Sac I and Sal I at 37° C. for 1 hour. Then, generation of bands was confirmed through electrophoresis. The nucleotide sequence analysis was performed on the candidate groups to confirm insertion of the flhDC gene.

Accordingly, flgM-IFN-β-flhDC-pBAD18-asd+plasmids according to Examples and Comparative Examples were prepared.

Meanwhile, the genetic information of major structures in this Example is as shown in Table 2.

TABLE 2

| | | SEQ ID |
|---|---|---|
| Name | Sequence information | NO: |
| flgM | atgagcattgaccgtacctcacctttgaaacccgttagcactgtccagacgcgcgaa | 1 |
| | accagcgacacgccggtacaaaaaacgcgtcaggaaaaaacgtccgccgcgac | |
| | gagcgccagcgtaacgttaagcgacgcgcaagcgaagctcatgcagccaggcgt | |
| | cagcgacattaatatggaacgcgtcgaagcattaaaaacggctatccgtaacggtg | |
| | agttaaaaatggatacgggaaaaatagcagactcgctcattcgcgaggcgcagag | |
| | ctacttacagagtaaaaaa | |
| IFN-β1 | ATGATTAATTATAAACAGCTGCAGCTGCAGGAACGTACCA | 2 |
| | ATATTCGTAAATGTCAGGAACTGCTGGAACAGCTGAATGG | |
| | TAAAATTAATCTGACCTATCGTGCAGATTTTAAAATTCCGA | |
| | TGGAAATGACCGAAAAAATGCAGAAAAGCTATACCGCATT | |
| | TGCCATTCAGGAAATGCTGCAGAATGTTTTTCTGGTTTTT | |
| | CGTAATAATTTTAGCAGCACCGGTTGGAATGAAACCATTG | |
| | TTGTTCGTCTGCTGGATGAACTGCATCAGCAGACCGTTTT | |
| | TCTGAAAACCGTTCTGGAAGAAAAACAGGAAGAACGTCT | |
| | GACCTGGGAAATGAGCAGCACCGCACTGCATCTGAAAAG | |
| | CTATTATTGGCGTGTTCAGCGTTATCTGAAACTGATGAAA | |
| | TATAATAGCTATGCCTGGATGGTGGTTCGTGCAGAAATTT | |
| | TTCGTAATTTTCTGATTATTCGTCGTCTGACCCGTAATTTT | |
| | CAGAATTAG | |
| IFN-β2 | ATGATTAACTATAAGCAGCTGCAGCTGCAGGAACGTACTA | 3 |
| | ACATCCGTAAATGCCAGGAACTTCTTGAACAGCTGAACG | |
| | GTAAAATCAACCTGACCTACCGTGCGGACTTCAAAATCCC | |
| | CATGGAAATGACGGAAAAAATGCAGAAATCTTACACCGC | |
| | GTTCGCGATCCAGGAAATGCTGCAGAACGTTTTCCTGGT | |
| | TTTCCGTAACAACTTCTCTTCTACCGGTTGGAACGAAACC | |
| | ATCGTTGTTCGTCTGCTGGACGAACTGCACCAGCAGACC | |
| | GTTTTCCTGAAAACCGTTCTGGAAGAAAAACAGGAAGAAC | |
| | GTCTGACCTGGGAAATGTCTAGCACCGCGCTGCACCTGA | |
| | AATCTTACTACTGGCGTGTTCAGCGTTACCTGAAACTGAT | |
| | GAAATACAACTCTTACGCGTGGATGGTTGTTCGTGCGGA | |
| | AATCTTCCGTAACTTCCTGATCATCCGTCGTCTGACCCGT | |
| | AACTTCCAGAACTAA | |
| Linker 1 | GGCGGCAGCAGCCATCACCATCACCATCACAGCAGCGG | 4 |
| | CGGCTTAGTTCCGCGCGGTAGCCAT | |
| Linker 2 | ggcggcagcagccatcaccatcaccatcacagcagcggcggc | 5 |
| flhDC | AGGAGGTTTGATCCTatgggaacaatgcatacatccgagttgctaaaaca | 7 |
| | catttatgacatcaatttgtcatatttactccttgcacagcgtttgatcgtccaggacaaa | |
| | gcatctgcgatgttccgcctcggtatcaacgaagagatggcaaacacactgggcgc | |
| | gttgaccctgccgcagatggtcaaactggcggagacgaaccagttagtttgtcatttc | |
| | cggtttgacgatcatcagacgatcacccgtttgactcaggattcgcgcgtcgatgactt | |
| | acagcagattcacacaggtatcatgctttcaacgcgtctgctcaatgaagtggacgat | |
| | acggcgcgtaagaaaagggcatgaaagggcatgataatgagtgaaaaaagcatt | |
| | gttcaggaagctcgcgatatccagttggcgatggagttgattaatcttggcgctcgtcta | |
| | caaatgctggaaagcgaaacacagctcagccgtggtcgcctcatcaggctgtaca | |
| | aagaattacgcggtagcccgccgcctaaagggatgctgccattttcgacagactggt | |
| | ttatgacctgggagcaaaatattcatgcctccatgttctgcaacgcctggcaatttttact | |
| | gaagaccggcttatgcagcggtgtggatgcggtgattaaagcttatcggctttatcttg | |
| | agcagtgtccgcaaccgcctgaagggccgttgttggcgctgactcgcgcatggacg | |
| | ctggtgcgttttgttgaaagtgggttgcttgaattgtcgagctgtaactgctgcggtggga | |
| | actttattacccatgcgcatcagcccgtaggcagctttgcgtgtagtttatgccagccgc | |
| | catcccgcgcagtaaaaagacgtaaactttcccgagatgctgccgatattattccaca | |
| | actgctggatgaacagatcgaacaggctgtttaa | |

### 1-3. Example 2: Preparation of Attenuated Salmonella Strain Secreting flgM-IFN-3

An attenuated Salmonella strain was transformed with the plasmid prepared in Example 1-2. For use as the attenuated Salmonella strain, a BRD509 asd galE:tetRA-strain (#1317) and a BRD509 asd rcsB galE:tetRA-strain (#1323) were used, each of which was provided by Chungnam National University. The genetic information and strain information of the transformed strains are as shown in Table 3.

TABLE 3

| | | Comparative | Comparative | Comparative |
|---|---|---|---|---|
| | Example 1 | Example 1 | Example 2 | Example 3 |
| IFN-β gene | IFN-β2 | IFN-β1 | IFN-β1 | IFN-β2 |
| Linker gene | Linker 2 | Linker 1 | Linker 1 | Linker 2 |
| Strain | #1323 | #1317 | #1323 | #1317 |

The inventors of the present disclosure deposited the flgM-IFN-3-flhDC-pBAD18-asd+/BRD509 asd rcsB galE:tetRA-strain according to an embodiment to the Korean Collection for Type Cultures of LBiological Resource Center of the Korea Research Institute of Bioscience and Biotechnology on Jul. 18, 2023, and the strain was assigned the Accession No: KCTC15509BP (S-flgM-mIFNb2-flhDC-pBAD18-ASD+/#1323).

### Experimental Example 1. Confirmation of Ability to Express and Secrete Protein

To confirm the ability of the attenuated Salmonella strain transformed with the recombinant expression vector prepared according to an embodiment for the expression and secretion of the flgM-IFN-β, the expression level of the protein was measured through western blotting by the following methods.

Specifically, a single colony of each of the transformed strains was inoculated into a LM amp liquid medium and cultured with shaking at 37° C. for 12 to 16 hours. Afterwards, the culture was diluted in a fresh LB amp liquid medium until OD600 reached 0.05, and then cultured with shaking at 37° C. for 2 hours. Next, when the OD600 reached 0.4 to 0.6, a group treated with arabinose at a concentration 0.2% (w/v) (final) was classified as a test group(+), and a group not treated with arabinose was classified as a control group(−), and the groups were cultured with shaking at 37° C. for additional 5 hours. Then, samples of the test group and the control group were separated into a whole cell culture solution, a pellet, and a supernatant. The pellet and the supernatant were obtained by separation through centrifugation on the whole cell culture solution at 8,000 rpm. In addition, the supernatant was filtered through a 0.2 μm filter to completely remove bacteria, and the filtrate was concentrated by using the Pall Nanosep (OD010C35 3K omega).

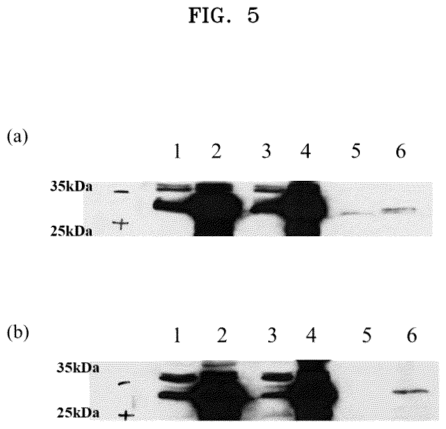

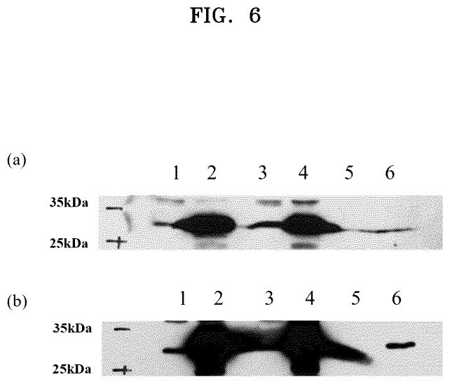

50 μl of each sample of the whole cell culture solution, the pellet, and the supernatant was mixed with an SDS sample buffer to prepare samples for Western blotting. Each sample was denatured in a 100° C. heating block for 5 minutes, centrifuged at 13,000 rpm and 4° C. for 5 minutes, and electrophoresed on a 10% polyacrylamide gel. The gel was transferred by using polyvinylidene fluoride (PVDF) to transfer the protein onto a PVDF membrane. The membrane was then blocked with a blocking buffer at room temperature for 1 hour. Afterwards, anti-his tag (SB194b, Southern Biotech) as a primary antibody was diluted at 1/1000 and reacted at 4° C. for 16 hours. Afterwards, the membrane was washed three times with tris-buffered saline with 0.1% Tween-20 (TBST) at 10 minute-intervals, and then allowed for a reaction with the anti-mouse-IgG (#7076s, Cell signaling, Danvers, MA, USA) secondary antibody at room temperature for 1 hour. Following the reaction, the membrane was washed with TBST at 10 minute-intervals, sensitized to an X-ray film by using an ECL buffer, and then developed to confirm the expression level of each protein. The results are shown in FIGS. 5 and 6. In FIGS. 5 and 6, lanes 2, 4, and 6 show the results for the test groups treated with the arabinose (final concentration of 0.2% (w/v)), and lanes 1, 3, and 5 show the results for the arabinose-untreated groups. In addition, in FIGS. 5 and 6, lanes 1 and 2 show the results for the whole cell culture solution, lanes 3 and 4 show the results for the pellet, and lanes 5 and 6 show the results for the supernatant.

Consequently, as shown in FIGS. 5 and 6, when the strain according to an embodiment was used, the secretion of the protein was confirmed even in the supernatant from which the strain was removed. In addition, it was confirmed that the strain according to an embodiment not only expressed the flgM-IFN-β at a high level, but also secreted the flgM-VC1 outside the strain at a high level ((b) of FIG. 6).

### Experimental Example 2. Confirmation of Antitumor Effects

To confirm the anticancer effects of the strain according to an embodiment, the following experiment was performed.

### 2-1. Preparation of Tumor Cell Line

Murine CT26 colon carcinoma cells were purchased from the American Type Culture Collection. The cells were cultured in a DMEM supplemented with 10% FBS and 1% penicillin-streptomycin.

### 2-2. Preparation of Mouse Model with Xenografted Tumor Cells

A tumor mouse model was prepared by xenografting the CT26 colon cancer cells cultured in Experimental Example 2-1. All animal care, experiments, and euthanasia were performed according to the approved protocols. Mice used herein were 6-week-old female mice weighing about 20 g and purchased from OrientBio.

Specifically, CT26 tumor cells cultured in vitro as in Experimental Example 2-1 were collected and suspended in 20 μl of PBS. 5×10⁵ of the suspended cells were injected subcutaneously into the right back of a BALB/c mouse to perform xenografting of the tumor cells. Afterwards, when the tumor size reached 100 mm³ (about 110 to 14 days after xenografting of the tumor cells), the strain according to an embodiment was treated.

### 2-3. Confirmation of Antitumor Effects

To confirm the anticancer effect and stability of the strain according to an embodiment, the following experiment was performed.

Specifically, for the tumor mouse model of Experimental Example 2-2, the experiment was carried by dividing the mouse models into i) a group treated with PBS as a control group (referred to as ctrl), ii) a group treated with the strain of Example 1 and but not treated with arabinose (referred to as Bacteria), and iii) a group treated with the strain of Example 1 and then arabinose (referred to as induction). Here, the strain of Example 1 was administered (Day 0) via the tail vein at a dose of about 1×10⁷ CFU/mouse in PBS. Regarding iii) the arabinose-treated group, L-arabinose was administered intraperitoneally (i.p.) daily at 80 mg/mouse to induce expression of IFN-β starting 3 days after the strain injection. Tumor size was measured once every two days, and the body weight was measured once every two days. Tumor volume was calculated as follows: ‘tumor volume (mm³)=(tumor length×tumor height×tumor width)/2’. All animal experiments were approved by the Animal Experiment Ethics Committee of Chungnam National University, and mice having a tumor volume of 1,500 mm³ or more were sacrificed according to the guidelines.

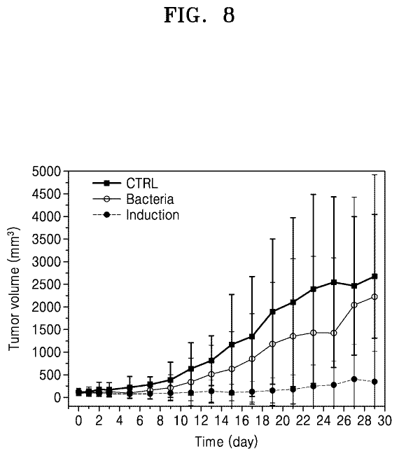

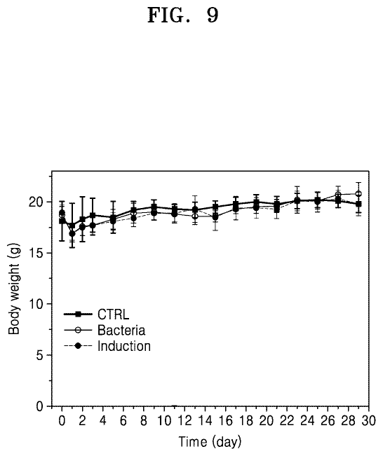

Consequently, as shown in FIG. 8, it was confirmed that the tumor size was significantly reduced in the arabinose-induced group, confirming the excellent anticancer effects of the attenuated Salmonella strain according to an embodiment. In addition, as shown in FIG. 9, no change was observed in the body weight of mice in the arabinose-induced group, confirming stability of the preparation including the strain according to the present disclosure.

While the foregoing has described certain aspects of the present disclosure, it will be apparent to those skilled in the art that these specific techniques are merely preferred embodiments and are not intended to limit the scope of the present disclosure. Therefore, the substantial scope of the present disclosure will be defined by the appended claims and equivalents thereof.

### Accession No

Name of depository institution: Korean Collection for Type Cultures (KCTC) of Korea Research Institute of Bioscience and Biotechnology
Accession No: KCTC15509BP
Accession Date: 20230718

1. A recombinant expression vector comprising: an flgM gene; an IFN-β gene; and an flhDC gene, wherein the flgM gene, the IFN-β gene, and the flhDC gene are operably linked to an inducible promoter.

2. The recombinant expression vector of claim 1, wherein expression of the flgM gene, the IFN-β gene, and the flhDC gene is regulated by the same inducible promoter.

3. The recombinant expression vector of claim 1, wherein the recombinant expression vector comprises the flgM gene consisting of a polynucleotide sequence of SEQ ID NO: 1, the IFN-β gene consisting of a polynucleotide sequence of SEQ ID NO: 3, and the flhDC gene consisting of a polynucleotide sequence of SEQ ID NO: 7.

4. The recombinant expression vector of claim 1, wherein the flgM gene and the IFN-β gene are linked via a linker.

5. The recombinant expression vector of claim 4, wherein the linker consists of a polynucleotide sequence of SEQ ID NO: 5.

6. An attenuated Salmonella strain transformed with the recombinant expression vector of claim 1.

7. The attenuated Salmonella strain of claim 6, wherein the attenuated Salmonella strain has loss of function of an aroA gene, an aroD gene, an asd gene, an rcsB gene, and a galE gene.

8. The attenuated Salmonella strain of claim 6, wherein the attenuated Salmonella strain has an enhanced ability to secrete an IFN-β protein.

9. The attenuated Salmonella strain of claim 6, wherein the attenuated Salmonella strain is deposited with Accession No. KCTC15509BP.

10. A pharmaceutical composition for cancer treatment, comprising the attenuated Salmonella strain of claim 6 as an active ingredient.


SEQUENCE LISTING (10 sequences)

SEQ ID NO: 1 - DNA, 294 bp, synthetic construct
  source 1..294: mol_type = other DNA
atgagcattgaccgtacctcacctttgaaacccgttagcactgtccagacgcgcgaaaccagcgacacgccggtacaaaaaacgcgtcaggaaaaaacgtccgccgcgacgagcgccagcgtaacgttaagcgacgcgcaagcgaagctcatgcagccaggcgtcagcgacattaatatggaacgcgtcgaagcattaaaaacggctatccgtaacggtgagttaaaaatggatacgggaaaaatagcagactcgctcattcgcgaggcgcagagctacttacagagtaaaaaa

SEQ ID NO: 2 - DNA, 489 bp, synthetic construct
  source 1..489: mol_type = other DNA
atgattaattataaacagctgcagctgcaggaacgtaccaatattcgtaaatgtcaggaactgctggaacagctgaatggtaaaattaatctgacctatcgtgcagattttaaaattccgatggaaatgaccgaaaaaatgcagaaaagctataccgcatttgccattcaggaaatgctgcagaatgtttttctggtttttcgtaataattttagcagcaccggttggaatgaaaccattgttgttcgtctgctggatgaactgcatcagcagaccgtttttctgaaaaccgttctggaagaaaaacaggaagaacgtctgacctgggaaatgagcagcaccgcactgcatctgaaaagctattattggcgtgttcagcgttatctgaaactgatgaaatataatagctatgcctggatggtggttcgtgcagaaatttttcgtaattttctgattattcgtcgtctgacccgtaattttcagaattag

SEQ ID NO: 3 - DNA, 489 bp, synthetic construct
  source 1..489: mol_type = other DNA
atgattaactataagcagctgcagctgcaggaacgtactaacatccgtaaatgccaggaacttcttgaacagctgaacggtaaaatcaacctgacctaccgtgcggacttcaaaatccccatggaaatgacggaaaaaatgcagaaatcttacaccgcgttcgcgatccaggaaatgctgcagaacgttttcctggttttccgtaacaacttctcttctaccggttggaacgaaaccatcgttgttcgtctgctggacgaactgcaccagcagaccgttttcctgaaaaccgttctggaagaaaaacaggaagaacgtctgacctgggaaatgtctagcaccgcgctgcacctgaaatcttactactggcgtgttcagcgttacctgaaactgatgaaatacaactcttacgcgtggatggttgttcgtgcggaaatcttccgtaacttcctgatcatccgtcgtctgacccgtaacttccagaactaa

SEQ ID NO: 4 - DNA, 63 bp, synthetic construct
  source 1..63: mol_type = other DNA
ggcggcagcagccatcaccatcaccatcacagcagcggcggcttagttccgcgcggtagccat

SEQ ID NO: 5 - DNA, 42 bp, synthetic construct
  source 1..42: mol_type = other DNA
ggcggcagcagccatcaccatcaccatcacagcagcggcggc

SEQ ID NO: 6 - DNA, 825 bp, synthetic construct
  source 1..825: mol_type = other DNA
atgagcattgaccgtacctcacctttgaaacccgttagcactgtccagacgcgcgaaaccagcgacacgccggtacaaaaaacgcgtcaggaaaaaacgtccgccgcgacgagcgccagcgtaacgttaagcgacgcgcaagcgaagctcatgcagccaggcgtcagcgacattaatatggaacgcgtcgaagcattaaaaacggctatccgtaacggtgagttaaaaatggatacgggaaaaatagcagactcgctcattcgcgaggcgcagagctacttacagagtaaaaaaggcggcagcagccatcaccatcaccatcacagcagcggcggcatgattaactataagcagctgcagctgcaggaacgtactaacatccgtaaatgccaggaacttcttgaacagctgaacggtaaaatcaacctgacctaccgtgcggacttcaaaatccccatggaaatgacggaaaaaatgcagaaatcttacaccgcgttcgcgatccaggaaatgctgcagaacgttttcctggttttccgtaacaacttctcttctaccggttggaacgaaaccatcgttgttcgtctgctggacgaactgcaccagcagaccgttttcctgaaaaccgttctggaagaaaaacaggaagaacgtctgacctgggaaatgtctagcaccgcgctgcacctgaaatcttactactggcgtgttcagcgttacctgaaactgatgaaatacaactcttacgcgtggatggttgttcgtgcggaaatcttccgtaacttcctgatcatccgtcgtctgacccgtaacttccagaactaa

SEQ ID NO: 7 - DNA, 957 bp, synthetic construct
  source 1..957: mol_type = other DNA
aggaggtttgatcctatgggaacaatgcatacatccgagttgctaaaacacatttatgacatcaatttgtcatatttactccttgcacagcgtttgatcgtccaggacaaagcatctgcgatgttccgcctcggtatcaacgaagagatggcaaacacactgggcgcgttgaccctgccgcagatggtcaaactggcggagacgaaccagttagtttgtcatttccggtttgacgatcatcagacgatcacccgtttgactcaggattcgcgcgtcgatgacttacagcagattcacacaggtatcatgctttcaacgcgtctgctcaatgaagtggacgatacggcgcgtaagaaaagggcatgaaagggcatgataatgagtgaaaaaagcattgttcaggaagctcgcgatatccagttggcgatggagttgattaatcttggcgctcgtctacaaatgctggaaagcgaaacacagctcagccgtggtcgcctcatcaggctgtacaaagaattacgcggtagcccgccgcctaaagggatgctgccattttcgacagactggtttatgacctgggagcaaaatattcatgcctccatgttctgcaacgcctggcaatttttactgaagaccggcttatgcagcggtgtggatgcggtgattaaagcttatcggctttatcttgagcagtgtccgcaaccgcctgaagggccgttgttggcgctgactcgcgcatggacgctggtgcgttttgttgaaagtgggttgcttgaattgtcgagctgtaactgctgcggtgggaactttattacccatgcgcatcagcccgtaggcagctttgcgtgtagtttatgccagccgccatcccgcgcagtaaaaagacgtaaactttcccgagatgctgccgatattattccacaactgctggatgaacagatcgaacaggctgtttaa

SEQ ID NO: 8 - DNA, 58 bp, synthetic construct
  source 1..58: mol_type = other DNA
gatcgatcgagctcaggaggtttgatcctatgggaacaatgcatacatccgagttgct

SEQ ID NO: 9 - DNA, 49 bp, synthetic construct
  source 1..49: mol_type = other DNA
gatcgatcgtcgacttaaacagcctgttcgatctgttcatccagcagtt

SEQ ID NO: 10 - DNA, 1107 bp, synthetic construct
  source 1..1107: mol_type = other DNA
atgaaaaatgttggttttatcggctggcgcggaatggtcggctctgttctcatgcaacgcatggtagaggagcgcgatttcgacgctattcgccctgttttcttttctacctcccagtttggacaggcggcgcccaccttcggcgacacctccaccggcacgctacaggacgcttttgatctggatgcgctaaaagcgctcgatatcatcgtgacctgccagggcggcgattataccaacgaaatttatccaaagctgcgcgaaagcggatggcagggttactggattgatgcggcttctacgctgcgcatgaaagatgatgccattattattctcgacccggtcaaccaggacgtgattaccgacggcctgaacaatggcgtgaagacctttgtgggcggtaactgtaccgttagcctgatgttgatgtcgctgggcggtctctttgcccataatctcgttgactgggtatccgtcgcgacctatcaggccgcctccggcggcggcgcgcgccatatgcgcgagctgttaacccagatgggtcagttgtatggccatgtcgccgatgaactggcgacgccgtcttccgcaattcttgatattgaacgcaaagttacggcattgacccgcagcggcgagctgccggttgataactttggcgtaccgctggcgggaagcctgatcccctggatcgacaaacagctcgataacggccagagccgcgaagagtggaaaggccaggcggaaaccaacaagattctcaatactgcctctgtgattccggttgatggtttgtgtgtgcgcgtcggcgcgctgcgctgtcacagccaggcgttcaccatcaagctgaaaaaagaggtatccattccgacggtggaagaactgctggcggcacataatccgtgggcgaaagtggtgccgaacgatcgtgatatcactatgcgcgaattaaccccggcggcggtgaccggcacgttgactacgccggttggtcgtctgcgtaagctgaacatggggccagagttcttgtcggcgtttaccgtaggcgaccagttgttatggggcgccgccgagccgctgcgtcgaatgctgcgccagttggcgtag